(19) United States
(12) Patent Application Publication
(10) Pub. No.: US 2017/0086671 A1
(43) Pub. Date: Mar. 30, 2017

(54) INTELLIGENT ORTHOPEDIC APPARATUS, SYSTEMS, AND METHODS

(71) Applicant: Zimmer, Inc., Warsaw, IN (US)

(72) Inventor: Randy Sessler, Warsaw, IN (US)

(21) Appl. No.: 15/273,827

(22) Filed: Sep. 23, 2016

Related U.S. Application Data
(60) Provisional application No. 62/232,787, filed on Sep. 25, 2015.

(51) Int. Cl. A61B 5/00; A61B 5/11; A61F 5/01

(52) U.S. Cl. CPC A61B 5/0022; A61F 5/0123; A61F 5/0109; A61B 5/1121; A61F 2005/0188; A61F 2005/0134

(57) ABSTRACT

A system can comprise an orthopedic sleeve, a processor, and a database. The orthopedic sleeve can be configured to be worn over a joint of a user and can comprise at least one sensor and a communicator. The at least one sensor can be operable to monitor at least one activity parameter associated with the joint, and the communicator can be configured to communicate activity data associated with the at least one activity parameter of the joint. The processor can be configured to receive the activity data from the communicator, and the database in communication with the processor can be configured to store the activity data. Additional apparatus, methods, and systems are disclosed.

### PRIORITY APPLICATIONS

This application claims the benefit of priority to U.S. Provisional Application Ser. No. 62/232,787, filed Sep. 25, 2015, the content of which is incorporated herein in its entirety.

### BACKGROUND

Human joints are capable of a number of movements. For example, some joints may experience flexion, extension, hyperextension, abduction, adduction, circumduction, rotation, retraction, protraction, elevation, depression, etc. As a result of these various movements, joints are often exposed to a number of forces that may lead to injury, and may even require surgery. Following injury or surgery, the performance of the joint may be altered. Sometimes the changes in the performance of the joint after injury or surgery are tracked through physical therapy.

### Overview

To better illustrate the instrument disclosed herein, a non-limiting list of examples is provided here:

In Example 1, an apparatus can be provided that includes an orthopedic sleeve configured to be worn over a joint of a user and at least one sensor attached to the orthopedic sleeve, the at least one sensor operable to monitor at least one activity parameter associated with the joint.

In Example 2, the apparatus of Example 1 is optionally configured such that the activity parameter is range of motion of the joint.

In Example 3, the apparatus of Example 1 is optionally configured such that the activity parameter is force or load on the joint.

In Example 4, the apparatus of Example 1 is optionally configured such that the activity parameter is stress or strain on the joint.

In Example 5, the apparatus of Example 1 is optionally configured such that the activity parameter is displacement of at least one bone of the joint.

In Example 6, the apparatus of any one of or any combination of Examples 1-5 is optionally configured such that the at least one sensor is selected from the group consisting of: an accelerometer, a gyrometer, a position sensor, a force sensor, a strain gauge, a pressure sensor, and a torque sensor.

In Example 7, the apparatus of any one of or any combination of Examples 1-6 is optionally configured such that the at least one sensor is embedded within the sleeve.

In Example 8, the apparatus of any one of or any combination of Examples 1-7 is optionally configured such that the orthopedic sleeve comprises an orthopedic brace.

In Example 9, the apparatus of any one of or any combination of Examples 1-8 is optionally configured such that the apparatus includes a transmitter attached to the orthopedic sleeve configured to transmit a signal received from the at least one sensor.

In Example 10, a system can be provided that includes an orthopedic sleeve configured to be worn over a joint of a user that includes at least one sensor configured to monitor at least one activity parameter associated with the joint and a communicator configured to communicate activity data associated with the at least one activity parameter of the joint. The system can also include a processor configured to receive the activity data from the communicator and a database in communication with the processor, the database configured to store the activity data of the user.

In Example 11, the system of Example 10 is optionally configured such that the communicator comprises a transmitter or transceiver.

In Example 12, the system of any one of or any combination of Examples 10 and 11 is optionally configured such that the communicator comprises a connector to electrically couple the sensor to the processor.

In Example 13, the system of any one of or any combination of Examples 10-12 is optionally configured such that the system includes a smartphone comprising the processor.

In Example 14, a method can be provided that includes monitoring, via at least one sensor attached to an orthopedic sleeve, at least one activity parameter associated with a joint of a user, communicating activity data associated with the at least one activity parameter to a processor, and storing the activity data at a database.

In Example 15, the method of Example 14 optionally includes providing diagnostic information based on the activity data.

In Example 16, the method of any one of or any combination of Examples 14 and 15 optionally includes providing instructions for the user based on the at least one activity parameter.

In Example 17, the method of any one of or any combination of Examples 14-16 optionally includes post-surgical monitoring, pre-surgical monitoring, pre-injury monitoring, post-injury monitoring, or injury monitoring.

In Example 18, the method of any one of or any combination of Examples 14-17 optionally includes updating a smartphone application based on data associated with the at least one activity parameter.

In Example 19, the method of any one of or any combination of Examples 14-18 optionally includes displaying, at a user interface, analysis of the activity data.

In Example 20, the method of any one of or any combination of Examples 14-19 optionally includes transmitting instructions from the processor to the at least one sensor.

In Example 21, the apparatus, system, or method of any one or any combination of Examples 1-20 can optionally be configured such that all elements or options recited are available to use or select from.

These and other examples and features of the present devices, systems, and methods will be set forth in part in the following Detailed Description. This overview is intended to provide a summary of subject matter of the present patent application. It is not intended to provide an exclusive or exhaustive removal of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a block diagram of an intelligent orthopedic system including an orthopedic sleeve, in accordance with at least one example of the present disclosure.

FIG. 2 is a perspective view of an orthopedic sleeve, in accordance with at least one example of the present disclosure.

FIG. 3 is another perspective view of an orthopedic sleeve, in accordance with at least one example of the present disclosure.

FIG. 4 is a flow chart of an example method of using an intelligent orthopedic system, in accordance with at least one example of the present disclosure.

### DETAILED DESCRIPTION

An intelligent orthopedic system can monitor the performance of joints or other body parts to provide useful information about patients, injuries, treatment and rehabilitation methods, or the like. For example, monitoring the performance of a joint leading up to, or at the time of, injury can provide valuable insight into the cause or even the diagnosis of the injury, which may lead to a better understanding of preventative measures for that type of injury. In another example, an intelligent orthopedic system can be used following an injury to track progress of rehabilitation of a joint and determine an appropriate physical therapy regimen. In some examples, an intelligent orthopedic system can be used to monitor or otherwise assess performance of an implant. While many of the examples are described with reference to a knee joint, the apparatus and methods can similarly be applied to any joints or body parts.

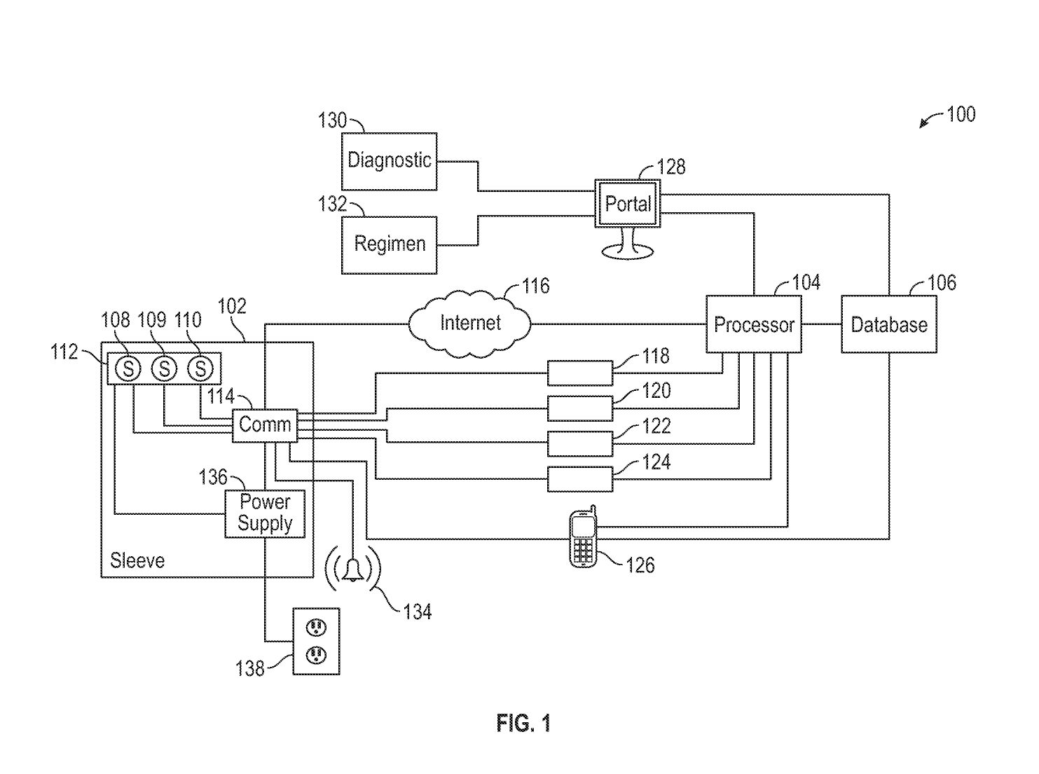

FIG. 1 is a block diagram of an intelligent orthopedic system 100, in accordance with at least one example of the present disclosure. The intelligent orthopedic system 100 can include an orthopedic sleeve 102, a processor 104, and a database 106. In some examples, the orthopedic sleeve 102 can be worn over a joint of a user, for example, the knee of a user. In other examples, the orthopedic sleeve 102 can be worn over the elbow, ankle, wrist, shoulder, hip, foot, neck, head, hand, or other body part of the user. In some examples, the orthopedic sleeve 102 can comprise a sleeve, a brace, a shoe, a helmet, a support, or the like.

The orthopedic sleeve 102 can include one or more sensors 108, 109, 110. In at least one example, at least two of the sensors 108, 109, 110 share a housing 112. The orthopedic sleeve 102 can include any number and variety of sensors 108, 109, 110. For example, each of the sensors 108, 109, 110 can comprise one or more of an accelerometer, a gyrometer, a position sensor, a force sensor, a strain gauge, a pressure sensor, a torque sensor, or the like. Each of the sensors 108, 109, 110 can be operable to monitor at least one activity parameter associated with a joint (or body part) of the user. For example, each of the sensors 108, 109, 110 can monitor one or more of: range of motion of the joint, force on the joint, load on the joint, stress on the joint, strain on the joint, displacement of at least one bone of a joint, or the like. In at least one example, the sensor includes a temperature sensor to indicate a temperature or a change in temperature that might suggest swelling or inflammation of the joint or other body part.

The orthopedic sleeve 102 can further include a communicator 114 in communication with the sensors 108, 109, 110. The communicator 114 can be configured to transmit a signal received from the one or more sensors 108, 109, 110. In some examples, the communicator 114 can be configured to communicate activity data associated with the activity parameter from the sensors 108, 109, 110 to the processor 104 or the database 106. The communicator 114 can comprise at least one of a transmitter and a transceiver. In some examples, the communicator 114 can comprise a connector to electrically couple the one or more sensors 108, 109, 110 to the processor 104. The communicator 114 can communicate date from the sensors 108, 109, 110 to the processor 104 synchronously or asynchronously using any of a variety of communication methods or formats. In some examples, the communicator 114 can communicate to the processor 104 via a wired connection, a wireless connection, or both. In some examples, the communicator 114 can communicate the data from the sensors 108, 109, 110 via one or more communication systems 116, 118, 120, 122, 124, 126. For example, the communicator 114 can communicate the data from the sensors 108, 109, 110 over the internet, via one or more ports, using near field communication, using Bluetooth technology, via radio waves, using a cellular data network, using a Wi-Fi network, using ZigBee, using one or more other communication protocols, via one or more smart devices, (e.g., a smartphone 126, a smartwatch, a tablet, a smart band, a smart key chain, a laptop, etc.), a combination of these, or the like. In some examples, the communicator 114 can communicate the data from the sensors 108, 109 110 to the processor 104 via two or more different communication systems 116, 118, 120, 122, 124, 126 at different times. For example, the communicator 114 can default to using a Wi-Fi connection if available, but if a Wi-Fi connection is unavailable, can use a cellular data network. In at least one example, the orthopedic sleeve 102 comprises a Wi-Fi source. In some examples, the communicator 114 can auto-connect to available networks, for example, public hot spots, Wi-Fi that does not require a password or for which the password is known, or the like.

In at least one example, the communicator 114 can communicate with the processor 104 via a communication network of peer-to-peer devices. For example, the communicator 114 can communicate with a nearby smart device which communicates with a second smart device, which communicates with a third smart device, which communicates with the processor via a shared network or other communication protocol. In at least one example, the smartphone 126 can comprise the processor 104. In at least one example, the communicator 114 can comprise multiple transmitters or multiple transceivers.

The processor 104 can be configured to receive activity data associated with the at least one activity parameter from the communicator 114. The processor 104 can store the activity data in the database 106. In at least one example, the processor 104 analyzes the activity data before storing information associated with the activity data in the database 106. The processor 104 can communicate with the database 106 via a wired or wireless connection, or both. In at least one example, the smartphone 126 can communicate the activity data or information associated with the activity data to the database 106.

In at least one example, the orthopedic sleeve 102 can be worn over a joint of a user to provide activity data to the processor 104 or the database 106 to aid in assessment of the performance of the joint. For example, the processor 104 can provide analysis of the activity data to a client portal 128 that can be accessed by the user or a representative of the user (for example, a physical therapist or other caregiver) to assess the performance of the joint. In at least one example, a diagnostic database 130 can provide diagnostic information that can be used in combination with the activity data to provide a diagnosis. For example, if a user wears the orthopedic sleeve following (or at the time of) an injury, the activity data of the joint can be analyzed based on the diagnostic information provided by the diagnostic database 130 to diagnose the injury. In some examples, the smartphone 126 can comprise the portal 128. For example, the user can view the activity data, or information based on the activity data on an application on the smartphone 126.

In some examples, the orthopedic sleeve 102 can be worn by the user during a period of rehabilitation following injury, surgery, or other trauma to the joint, such that the orthopedic sleeve 102 provides activity data to the processor 104 that can be used to determine the progress of rehabilitation of the joint. In at least one example, a regimen database 132 can provide exercise or physical therapy regimen information that can be used in combination with the activity data to determine a regimen for the user based on the rehabilitation status of the joint. For example, the regimen database 132 can comprise a variety of exercises associated with particular thresholds of a given activity parameter or activity data, such that each exercises is chosen based on the activity data of the user relative to the thresholds. In some examples, the user or a caregiver can view the regimen information on the portal 128. In at least one example, the smartphone 126 comprises the portal 128, such that the user can view the regimen information on an application on the smartphone 126.

In some examples, the orthopedic sleeve 102 can be worn by the user to assess an implant. For example, the orthopedic sleeve 102 can be used to measure the performance of the implant or to determine wear and tear or the life span of the implant. For example, the sleeve 102 can be worn by a user following a total knee replacement, and the intelligent orthopedic system 100 can provide information related to the performance of the implant during specific movements of the knee, such that a caregiver can determine if a subsequent modification of the implant is required. In at least one example, the orthopedic sleeve 102 can be used to compare the performance of different versions of implants, such that a surgeon or user can determine which implant to use based on the performance data of each implant provided by the intelligent orthopedic system 100. In at least one example, the intelligent orthopedic system 100 can be used to study what activities affect an implant's performance. For example, if total knee replacement patients who have worn the orthopedic sleeve 102 eventually require a revision surgery, the activity data collected by the orthopedic sleeve 102 can be used to determine what activities likely created the need for the revision surgery.

In some examples, the portal 128 can provide a live stream of the activity data, such that a user wearing orthopedic sleeve 102 can perform movements, and the activity data created by the movements is shown on the portal 128 in real time. For example, a physical therapist could have a user perform squats while wearing the orthopedic sleeve 102, and the portal 128 could display the activity data as the user is performing the squats, such that the physical therapist could modify the user's form, reduce the weight of the exercise, modify the user's regimen, determine the performance of an implant, determine if the knee is swelling, determine if the user needs to stop, a combination of these, or the like, as necessary based on the real time activity data.

In some examples, the sensors 108, 109, 110, the communicator 114, or both can have more than one mode, such as a synchronous mode and an asynchronous mode. In some examples, the synchronous mode is used to provide real time data to the portal 128. In at least one example, the synchronous mode involves a higher data transfer rate and greater power usage. In at least one example, the synchronous mode can be selected when connected to a communication system with sufficient bandwidth, for example, a good Wi-Fi source. In some examples, the synchronous mode can be selected when sufficient power is available. In some examples, the asynchronous mode can be used in a low power state and while connected to a poor bandwidth communication system. In some examples, the asynchronous mode can be the default, while the synchronous mode can be selected during certain activities or relevant assessment situations, e.g. while with a physical therapist, or the like.

In some examples, the sensors 108, 109, 110 can include memory (volatile or non-volatile). In at least one example, the sensors 108, 109, 110 can temporarily store sensor readings. In at least one example, while in asynchronous mode, the sensor 108, 109, 110 store data from the sensor readings in the memory until the communicator 114 can transmit the data. In at least one example, while in synchronous mode, the memory of the sensors 108, 109, 110 can facilitate a buffer to store and queue up the data for a high data rate transmission.

In some examples, the intelligent orthopedic system 100 can further include an alarm 134. The alarm 134 can be used to communicate that the joint has exceeded a predetermined activity parameter threshold. For example, a force threshold could be set to indicate that a user should not subject the joint to particular forces greater than the force threshold. In that case, if the user subjects the joint to a force greater than the force threshold, the alarm 134 can activate to indicate that the threshold has been exceeded. In some examples the communicator 114 communicates activity data to the alarm 134. In some examples, the orthopedic sleeve 102 comprises the alarm 134. In some embodiments, the smartphone 126, portal 128, or other device comprises the alarm 134. In some examples the alarm 134 is used to indicate a movement or exercise that should not be made based on how it affects the joint. In at least one example, the alarm 134 is used to facilitate injury prevention.

The orthopedic sleeve 102 can comprise a power supply 136 to power any of the sensors 108, 109, 110, the communicator 114, the alarm 134, other components, or a combination of these. In at least one example, the power supply 136 comprises batteries. The power supply 136 can comprise an active power supply or a passive power supply. In some examples, the orthopedic sleeve 102 can be plugged into an external outlet 138 to charge the power supply 136. In at least one embodiment, the power supply 136 charges while the orthopedic sleeve 102 is electrically coupled to the processor 104, the smartphone 126, or other electrical component. In some examples, the power supply 136 is kinetically charged by movement of the user while the user is wearing the orthopedic sleeve 102. In at least one example, the power supply 136 can be charged wirelessly, for example, using inductive charging via an induction charger. In some examples, the orthopedic sleeve 102 can include more than one power mode. In at least one example, the orthopedic sleeve 102 can include a low power mode in which some but not all of the features are used. For example, in a low power mode, the sensors 108, 109, 110 can collect data less frequently, less than all of the sensors 108, 109, 110 can be activated, the communicator 114 can communicate less frequently, a combination of these, or the like. In at least one example, the communicator 114 stops transmitting the activity data until the orthopedic sleeve 102 is taken out of lower power mode.

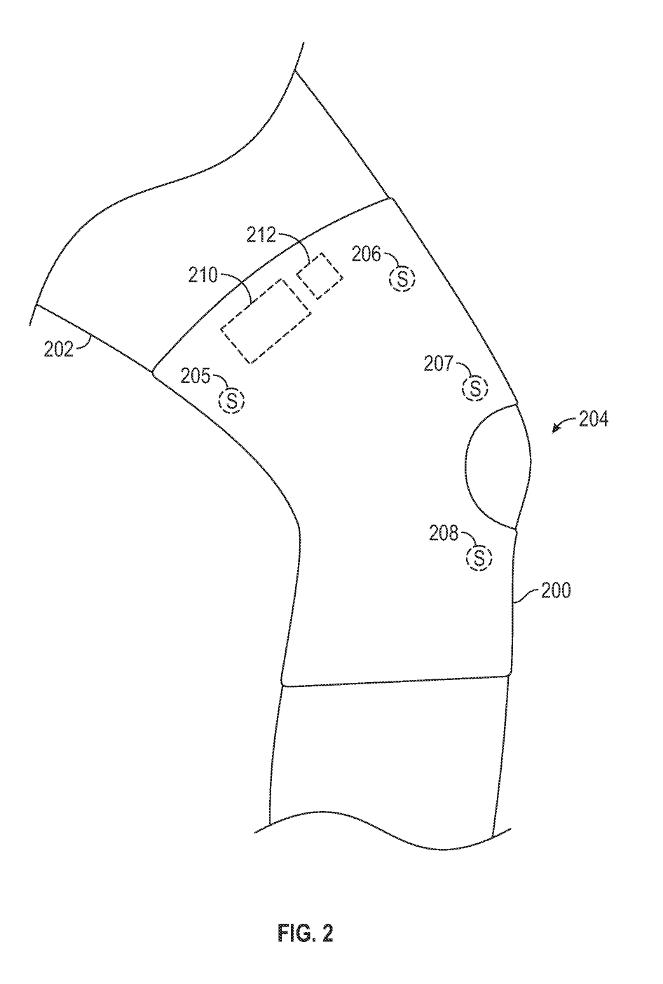

FIG. 2 is a perspective view of an orthopedic sleeve 200, in accordance with at least one example of the present disclosure. The orthopedic sleeve 200 can be configured to be worn by a user 202 over a joint 204 or other body part. In the illustrated example, the user 202 is wearing the orthopedic sleeve 200 over the knee 204. In some examples, the orthopedic sleeve 200 can be designed to be worn by the user 202 over a joint 204 other than the knee, for example, an elbow, a wrist, a shoulder, a finger, a neck, a hip, an ankle, a toe, or the like. In some examples the orthopedic sleeve 200 can be designed to be worn by the user 202 over a body part other than a joint, or in addition to a joint. In at least one embodiment, the orthopedic sleeve 200 can comprise a helmet to be worn by the user 202 over the head.

The orthopedic sleeve 200 can comprise any of a variety of materials, such as, plastic, metal, fabric, a combination of these, or the like. The orthopedic sleeve 200 can comprise at least one sensor (shown as four sensors in the illustrated example) 205, 206, 207, 208. In some examples the sensors 205, 206, 207, 208 can be attached to the orthopedic sleeve 200 on an outside surface or an interior surface. In some examples, the sensors 205, 206, 207, 208 can be embedded within the orthopedic sleeve 200. In various examples, the sensors 205, 206, 207, 208 can be placed in different locations relative to each other and the joint 204 of the user.

The sensors 205, 206, 207, 208 can each be operable to monitor at least one activity parameter associated with the joint 204. For example, each of the sensors 205, 206, 207, 208 can be operable to monitor range of motion, force, load, stress, strain, displacement, or the like. In some examples, each of the sensors 205, 206, 207, 208 can comprise an accelerometer, a gyrometer, a position sensor, a force sensor, a strain gauge, a pressure sensor, a torque sensor, or the like. For example, if the user 202 is recovering from an Anterior Cruciate Ligament (ACL) reconstruction surgery the orthopedic sleeve 200 can monitor activity of the knee joint 204 in order to facilitate monitoring rehabilitation, determining a physical therapy regimen, identifying activity of the user 202, modifying behavior of the user 202, or the like. For example, one or more of the sensors 205, 206, 207, 208 could monitor activity parameters that indicate an orientation of the knee joint 204 as the user 202 jumps. Using this information, a physical therapist, physician, or other caregiver could monitor the knee joint 204 even when the user 202 is not in a physical therapy setting, or in the presence of the caregiver, and identify if the user 202 has improper form, or is otherwise putting their knee joint 204 at risk. In at least one example, a smart device application could be used for the user 202 or other party to monitor the performance of the joint 204.

In some examples, the orthopedic sleeve 200 can comprise a power supply 210 to power one or more of the sensors 205, 206, 207, 208, a communicator 212, or other electrical components of the orthopedic sleeve 200. In some examples, the power supply 210 can comprise a rechargeable battery. The power supply 210 and the communicator 212 can each be embedded in the orthopedic sleeve 200, attached to the interior of the orthopedic sleeve 200, attached to the exterior of the orthopedic sleeve 200, or the like. In some examples, the sensors 205, 206, 207, 208 are (directly or indirectly) electrically coupled to the power supply 210 and the communicator 212. In some examples, the power supply 210 is kinetically charged by movement of the user 202 while the user 202 is wearing the orthopedic sleeve 200. In at least one example, the power supply 210 can be charged wirelessly, for example, using inductive charging via an induction charger. In some examples, the orthopedic sleeve 200 can include more than one power mode. In at least one example, the orthopedic sleeve 200 can include a low power mode in which some but not all of the features are used. For example, in a low power mode, the sensors 205, 206, 207, 208 can collect data less frequently, less than all of the sensors 205, 206, 207, 208 can be activated, a combination, or the like.

In some examples the communicator 212 can comprise one or more transmitters configured to transmit a signal received from the sensors 205, 206, 207, 208 to a processor for analysis or other processing and storage in a database. In some examples the communicator 212 can comprise one or more transceivers such that the communicator 212 can receive signals from the processor or other sources. For example, a caregiver or user 202 could decide to activate or deactivate some of the sensors 205, 206, 207, 208 in order to focus on particular activity of the joint 204 and could send instructions accordingly to the communicator 212. In some examples, the communicator 212 comprises a connector to electrically couple one or more of the sensors 205, 206, 207, 208 (directly or indirectly) to the processor. In some examples, the orthopedic sleeve 200 can be put in a low power mode or a limited communication mode in which the communicator 212 can communicate less frequently. In at least one example, the communicator 212 stops transmitting the activity data until the orthopedic sleeve 200 is taken out of low power mode.

In some examples, the communicator 212 can receive information about the user 202 from a smart device. For example, a user's smart phone might have a health application or other user input that includes information about the weight of the user 202 that could be transmitted to the communicator 212 to calibrate the sensors 205, 206, 207, 208 or otherwise tailor the activity data or analysis to the user. In at least one example, a smart device can be used to calibrate the orthopedic sleeve 200. For example, a smart device could count steps of the user 202, and the smart device step data could be compared to step data of the orthopedic sleeve 200 to calibrate the sensitivity or other parameters of the sensors 205, 206, 207, 208. In some examples, the comparison or calibration instructions could be performed by the smart device, the communicator 212, the processor, a combination of these, or the like.

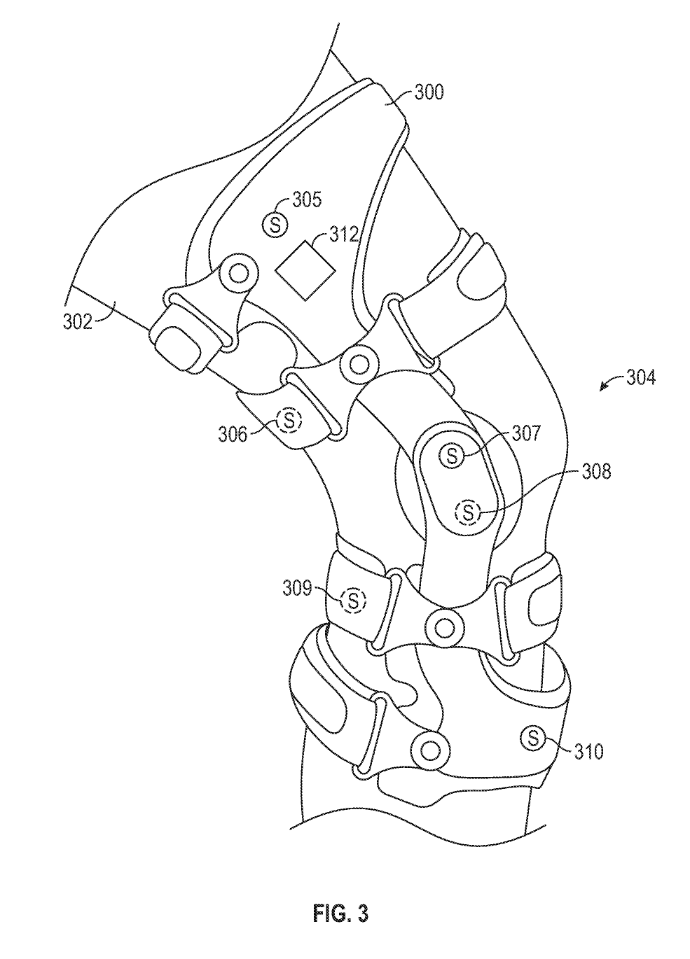

FIG. 3 is another perspective view of an orthopedic sleeve 300, in accordance with at least one example of the present disclosure. In the illustrated example, the orthopedic sleeve 300 is depicted as an orthopedic brace 300 worn by a user 302 over the knee joint 304. The orthopedic sleeve 300 can comprise any of a variety of materials, such as, plastic, metal, fabric, a combination of these, and the like. While the illustrated example depicts the orthopedic brace 300 as comprising six sensors 305, 306, 307, 308, 309, 310, other examples of the orthopedic sleeve 300 can comprise less sensors or more sensors, as long as the orthopedic sleeve 300 comprises at least one sensor. Each of the sensors 305, 306, 307, 308, 309, 310 can be attached to the orthopedic sleeve 300 in any of a variety of manners and locations. For example, one or more sensors 305, 307, 310 may be attached to the exterior of the orthopedic sleeve 300. As another example, one or more sensors 306, 308, 309 may be attached to the interior of the orthopedic sleeve 300 or embedded within the orthopedic sleeve 300. As described above, the sensors 305, 306, 307, 308, 309, 310 can be used to monitor and assess activity associated with the joint 304. In at least one example, one or more of the sensors 305, 306, 307, 308, 309, 310 can be used to monitor the performance of the orthopedic sleeve 300, or otherwise assess its condition. For example, one or more of the sensors 305, 306, 307, 308, 309, 310 can monitor activity parameters that can be used to identify that the orthopedic sleeve 300 is broken or improperly oriented.

In some examples, the orthopedic sleeve 300 can comprise a housing 312. The housing 312 can comprise, for example, a power supply, a communicator, a user interface, one or more of the sensors 305, 306, 307, 308, 309, 310, a controller, an activation switch, or the like. The housing 312 can be attached to an exterior surface of the orthopedic sleeve 300, attached to an interior surface of the orthopedic sleeve 300, or embedded within the orthopedic sleeve 300. In some examples, the housing 312 can comprise multiple housings in separate locations on the orthopedic sleeve 300.

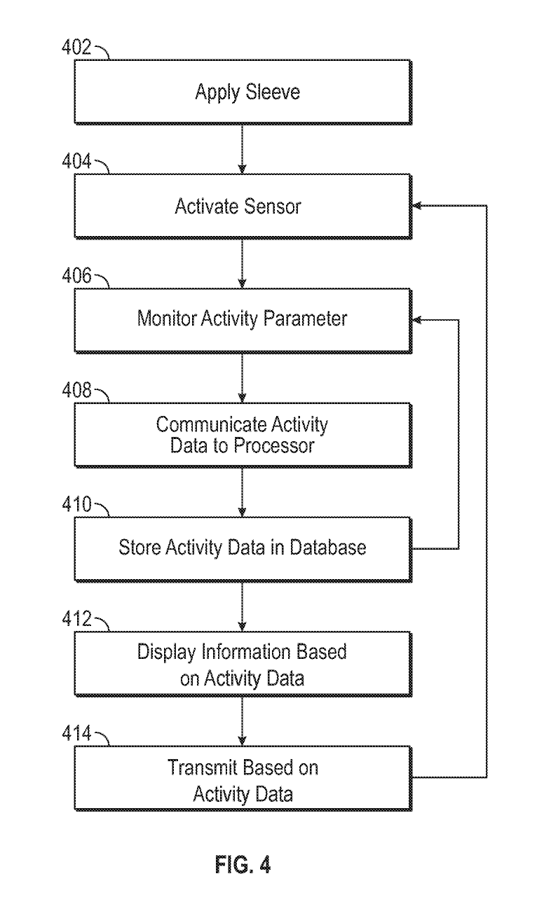

FIG. 4 is a flow chart of an example method 400 of using an intelligent orthopedic system, in accordance with at least one example of the present disclosure. As a matter of convenience, the method 400 is described with reference to the intelligent orthopedic system 100 of FIG. 1. At block 402, the user places the orthopedic sleeve 102 over, around, or proximate to a joint. For example, to monitor a knee joint of the user, the user can wear the orthopedic sleeve 102 over the user's knee. In some examples, the orthopedic sleeve 102 is placed over the user's joint by a physician or other caregiver. In some examples, the orthopedic sleeve 102 is configured to be worn over a user's body part other than a joint. In some examples, the orthopedic sleeve 102 can comprise a sleeve, a brace, a shoe, a helmet, a support, or the like.

At block 404, the user (or caregiver, or other party) can activate the at least one sensor 108, 109, 110 of the orthopedic sleeve 102. In at least one example, the user can activate the at least one sensor 108, 109, 110 by turning on the power supply 136. In some examples, the user can activate the at least one sensor 108, 109, 110 by sending instructions to the communicator 114. In at least one example, the orthopedic sleeve 102 can comprise multiple sensors 108, 109, 110, and the user can activate at least one of the sensors 108, 109, 110 while deactivating (or otherwise leaving deactivated) at least one of the sensors 108, 109, 110. In some examples, the user or caregiver can activate the at least one sensor 108, 109, 110 before the user or caregiver places the orthopedic sleeve 102 over the joint of the user. In some examples, the at least one sensor 108, 109, 110 can be activated by motion. In some examples, the at least one sensor 108, 109, 110 can be activated automatically at a predetermined time, or at predetermined intervals. In some examples, the sensor activation schedule can be adjusted. For example, the user can set the at least one sensor 108, 109, 110 to be active for five minutes of every hour between the hours of 8 a.m. and 5 p.m.

At block 406 the at least one sensor 108, 109, 110 can monitor at least one activity parameter associated with the activity of the user. For example, the at least one sensor 108, 109, 110, can comprise an accelerometer, a gyrometer, a position sensor, a force sensor, a strain gauge, a pressure sensor, a torque sensor, or the like. In the example of the orthopedic sleeve 102 monitoring a user's joint, the at least one sensor 108, 109, 110 can monitor range of motion of the joint, force on the joint, load on the joint, stress on the joint, strain on the joint, displacement of at least one bone of the joint, a combination of these, or the like. In some examples, the sensors 108, 109, 110 of the orthopedic sleeve 102 can be configured to monitor activity of a muscle, a bone, other body parts, or a combination of these. The user or caregiver can use the sensors 108, 109, 110 of the orthopedic sleeve 102 to facilitate post-surgical monitoring, pre-surgical monitoring, pre-injury monitoring, post-injury monitoring, or injury monitoring of the joint or other body part.

At block 408 the communicator 114 can communicate activity data associated with the activity parameter to the processor 104. In some examples, the communicator 114 can comprise a transmitter configured to receive a signal from the at least one sensor 108, 109, 110 and transmits the signal to the processor 104. In at least one example, the communicator 114 can comprise a transceiver. In at least one example, the activity data comprises data from the sensors 108, 109, 110 monitoring the at least one activity parameter. For example, if the at least one sensor 108, 109, 110 is monitoring range of motion of the knee joint, the at least one sensor 108, 109, 110 might send data indicative of the distance between the femur and the tibia (or fibula) at a given time, over a period of time, as a maximum value over a period of time, as a minimum value over a period of time, as an average value over a period of time, as a number or timestamp representing the value exceeding a threshold value, or the like. The communicator 114 can communicate the activity data to the processor 104 via the internet 116, via a port 118, using near field communication 120, using Bluetooth technology 122, via radio waves 124, via a smartphone 126, a combination of these, or the like.

At block 410 the processor 104 can store the activity data at the database 106. The database 106 maintains the activity data or related information for access by the user, or others. In some examples, the processor 104 analyzes the activity data and stores a resulting analysis at the database 106. In at least one example, the processor 104 can additionally be configured to be in electrical communication with a diagnostic database 130 to facilitate a diagnosis for the user based on the activity data from the orthopedic sleeve 102 and diagnostic data stored in the diagnostic database 130. In at least one example, the processor 104 can additionally be configured to be in electrical communication with a regimen database 132 to facilitate determination of an exercise or physical therapy regimen for the user based on the activity data from the orthopedic sleeve 102 and exercise or physical therapy data stored in the regimen database 132. In some examples, one or more of the monitoring of the activity parameters, communicating of activity data, and storing of activity data can continue even while the method 400 advances to block 412.

At block 412 the processor 104 can display information based on the activity data at the portal 128. In some examples, the processor 104 can display an analysis of the activity data at the portal 128. In at least one example, the processor 104 can display diagnostic information or exercise or physical therapy regimen information at the portal 128 based on the activity data associated with the joint. The portal 128 can comprise a computer, a smart device, a display, or the like. In some examples, the portal 128 provides a user interface for the user, a physician, a caregiver, or another to review or assess the activity of the user's joint.

At block 414 the processor 104 can transmit instructions to the communicator 114 based on the activity data. In some examples, the user (or other) can indicate instructions at the portal 128. In some examples, the processor 104 can send instructions to the communicator 114 based on an analysis of the activity data. In some examples, the processor 104 can send instructions to sound the alarm 134, which can be located at the client portal 128, at the orthopedic sleeve 102, or elsewhere. In some examples, the processor 104 can transmit instructions to activate (or deactivate) one or more of the at least one sensors 108, 109, 110, returning to block 404. In some examples, the processor 104 can transmit instructions to adjust one or more settings of one or more of the at least one sensors 108, 109, 110, the communicator 114 or the power supply 136. In at least one example, the processor 104 can provide instructions for the user based on the at least one activity parameter. In some examples, the processor 104 can transmit instructions to the at least one sensor 108, 109, 110. In at least one example, the processor 104 can update a smart device application based on data associated with the at least one activity parameter.

It should be noted that while certain features are described with regard to certain figures, many of the features from the different figures can be combined in a single embodiment.

In the foregoing Detailed Description, it can be seen that various features are grouped together in a single example for the purpose of streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus the following claims are hereby incorporated into the Detailed Description, with each claim standing on its own as a separate example.

Note that not all of the activities or elements described above in the general description are required, that a portion of a specific activity or device may not be required, and that one or more further activities may be performed, or elements included, in addition to those described. Still further, the order in which activities are listed are not necessarily the order in which they are performed. Also, the concepts have been described with reference to specific examples. However, one of ordinary skill in the art appreciates that various modifications and changes can be made without departing from the scope of the present disclosure as set forth in the claims below. Accordingly, the specification and figures are to be regarded in an illustrative rather than a restrictive sense, and all such modifications are intended to be included within the scope of the present disclosure.

Benefits, other advantages, and solutions to problems have been described above with regard to specific examples. However, the benefits, advantages, solutions to problems, and any feature(s) that may cause any benefit, advantage, or solution to occur or become more pronounced are not to be construed as a critical, required, or essential feature of any or all the claims. Moreover, the particular examples disclosed above are illustrative only, as the disclosed subject matter may be modified and practiced in different but equivalent manners apparent to those skilled in the art having the benefit of the teachings herein. No limitations are intended to the details of construction or design herein shown, other than as described in the claims below. It is therefore evident that the particular examples disclosed above may be altered or modified and all such variations are considered within the scope of the disclosed subject matter. Accordingly, the protection sought herein is as set forth in the claims below.

What is claimed is:

1. An apparatus, comprising:
an orthopedic sleeve configured to be worn over a joint of a user; and
at least one sensor attached to the orthopedic sleeve, the at least one sensor operable to monitor at least one activity parameter associated with the joint.

2. The apparatus of claim 1, wherein the activity parameter is range of motion of the joint.

3. The apparatus of claim 1, wherein the activity parameter is force or load on the joint.

4. The apparatus of claim 1, wherein the activity parameter is stress or strain on the joint.

5. The apparatus of claim 1, wherein the activity parameter is displacement of at least one bone of the joint.

6. The apparatus of claim 1, wherein the at least one sensor is selected from the group consisting of: an accelerometer, a gyrometer, a position sensor, a force sensor, a strain gauge, a pressure sensor, and a torque sensor.

7. The apparatus of claim 1, wherein the at least one sensor is embedded within the sleeve.

8. The apparatus of claim 1, wherein the orthopedic sleeve comprises an orthopedic brace.

9. The apparatus of claim 1, further comprising:
a transmitter attached to the orthopedic sleeve configured to transmit a signal received from the at least one sensor.

10. A system, comprising:
an orthopedic sleeve configured to be worn over a joint of a user, comprising:
at least one sensor configured to monitor at least one activity parameter associated with the joint; and
a communicator configured to communicate activity data associated with the at least one activity parameter of the joint;
a processor configured to receive the activity data from the communicator; and
a database in communication with the processor, the database configured to store the activity data of the user.

11. The system of claim 10, wherein the communicator comprises a transmitter or transceiver.

12. The system of claim 10, wherein the communicator comprises a connector to electrically couple the sensor to the processor.

13. The system of claim 10, further comprising:
a smartphone comprising the processor.

14. A method, comprising:
monitoring, via at least one sensor attached to an orthopedic sleeve, at least one activity parameter associated with a joint of a user;
communicating activity data associated with the at least one activity parameter to a processor; and
storing the activity data at a database.

15. The method of claim 14, further comprising:
providing diagnostic information based on the activity data.

16. The method of claim 14, further comprising:
providing instructions for the user based on the at least one activity parameter.

17. The method of claim 14, wherein the monitoring is selected from the group consisting of: post-surgical monitoring, pre-surgical monitoring, pre-injury monitoring, post-injury monitoring, and injury monitoring.

18. The method of claim 14, further comprising:
updating a smartphone application based on data associated with the at least one activity parameter.

19. The method of claim 14, further comprising:
displaying, at a user interface, analysis of the activity data.

20. The method of claim 14, further comprising:
transmitting instructions from the processor to the at least one sensor.
